(19) United States
(12) Patent Application Publication
(10) Pub. No.: US 2004/0265988 A1
(43) Pub. Date: Dec. 30, 2004

(54) Variovarax strains capable of degrading methyl tert-butyl either and their use

(76) Inventors: Jussi Uotila, Saarenkyla (FI); Gennadi Zaitsev, Rovaniemi (FI)

Correspondence Address: YOUNG & THOMPSON, 745 SOUTH 23RD STREET, 2ND FLOOR, ARLINGTON, VA, 22202, US

(21) Appl. No.: 10/492,935

(22) PCT Filed: Oct. 16, 2002
(86) PCT No.: PCT/FI02/00807

(30) Foreign Application Priority Data
Oct. 17, 2001 (FI) 20012016

(51) Int. Cl. C12N 1/20; C12N 1/00

(52) U.S. Cl. 435/243; 435/252.1; 435/262

(57) ABSTRACT

Water-soluble ethers used as octane number enhancers in gasoline are disturbing components in soils and groundwater even in small amounts due to their strong taste and odour. This invention relates to biological purification of gasoline-contaminated soils and groundwater, more specifically to Variovorax strains being able to degrade ethers and their degradation products and to a mixed bacterial population comprising said bacteria. Further this invention relates to a process for bacterial degradation of ethers and their degradation products and to the use of one or more Variovorax strains of the invention in purifying contaminated soil or water.

### FIELD OF THE INVENTION

The present invention relates to bacteria, which are capable of de-grading ethers and their degradation products. More precisely, the invention relates to Variovorax strain, to a mixed bacterial population and to a process for bacterial degradation of ethers and their degradation products. The invention further relates to the use of one or more of said strains in purifying contaminated soil and water.

### BACKGROUND

Ethers such as ethyl tert-butyl ether (ETBE), tert-amyl methyl ether (TAME) and methyl tert-butyl ether (MTBE) are widely used as octane number enhancers in unleaded gasoline. In Finland MTBE has been blended into gasoline at volumes of 9 to 13 vol.-%. MTBE, which replaced the lead in the 1980's in Europe, is compared to the aromatics, more effective as octane enhancer (fuel oxygenating agent) and safer to use from environmental perspectives. Also some tertiary alcohols, i.e. tert-butyl alcohol (TBA) and tert-amyl alcohol (TAA), are potential fuel oxygenates. TBA is an intermediate in the degradation of ETBE and MTBE.

Detection of MTBE in water supplies has focused attention on soil and groundwater contamination resulting from gasoline leaks and spills. There are different opinions on carcinogenity, teratogenity, mutagenity and neurotoxicity of MTBE. However, like other ethers, MTBE has a strong taste and odour and is thus detectable at very low levels of concentration, 35 μg 1⁻¹. Maximum concentration of MTBE in drinking water recommended by the U.S. EPA is 20 000 to 100 000 times lower than the lowest concentration that has caused observable health effects in animals. In certain hydrogeological conditions water soluble MTBE has a tendency to migrate in groundwater slightly faster and further than other gasoline components.

MTBE is a persistent substance in soil and groundwater. Private consumers can use activated carbon cartridges installed at the water tap as a temporary solution to remove the taste and odor of MTBE. However, neither MTBE nor other components of gasoline belong to soil or groundwater. Mechanical and/or chemical cleaning strategies in such large scales would be extremely troublesome and expensive, if not even impossible. Recent studies have shown the amazing ability of nature to bioremediate itself after, for example, oil disasters. Bioremediation is the process by which living organisms act to degrade hazardous organic contaminants or transform hazardous inorganic contaminant to environmentally safe levels in soils, subsurface materials, water and sludges. During the past decades researches have been conducted on the biodegradation potential of fuel oxygenating ethers and other gasoline components and biological clean-up strategies.

Salanitro et al., (Salanitro J. P., Diaz L. A., Williams M. P. and Wisniewski H. L. (1994) Appl. Environ. Microbiol. 60: 2593-2596) and U.S. Pat. No. 5, 750,346 were the first to demonstrate aerobic decomposition of MTBE by a mixed microbial culture at a rate of 34 mg per g of cells⁻¹ h⁻¹. TBA observed in the study was degraded even slower than MTBE. Anaerobic decomposition of MTBE was first reported by Mormille et al. (Mormille M. R., Liu S. and Suflita J. M. (1994) Environ. Sci. Technol. 28: 1727-1732). Anaerobic decomposition of MTBE is admittedly slow, but in many cases the situation can be improved by O₂ feed, nutrients and microbe augmentation.

Steffan et al. (Steffan R. J., McClay K., Vainberg S., Condee C. W. and Zhang D. (1997) Appl. Environ. Microbiol. 63: 4216-4222) suggest that MTBE is degraded by propane-oxidizing bacteria via tert-butoxy methanol to TBA by cleaving formaldehyde. TBA is further degraded via 2-methyl-2-hydroxy-1-propanol into 2-hydroxy isobutyric acid (HIBA), which is then degraded via 2-propanol, acetone and hydroxyacetone into pyruvic acid.

Hardison et al., (Hardison L. K., Curry S. S., Ciuffetti L. M. and Hyman M. R. (1997) Appl. Environ. Microbiol., 63: 3059-3067) demonstrated a filamentous fungus Graphium sp. strain ATCC 58400 which can cometabolically degrade low concentrations (750 ppb) of MTBE using diethyl ether as the source of carbon and energy. Tert-butyl formate (TBF) and TBA were detected as degradation products of MTBE. The kinetics of intermediate formation suggests that TBF production temporally precedes TBA accumulation and that TBF is hydrolyzed both biotically and abiotically to yield TBA.

Hanson et al. (Hanson J. R., Ackerman C. E. and Scow K. M. (1999) Appl. Environ. Microbiol., 65: 4788-4792) isolated a bacterial strain PM1 which is able to utilize MTBE as its sole carbon and energy source. Initial linear rates of MTBE degradation by 2×10⁶ cells ml⁻¹ were 0.07, 1.17 and 3.56 μg ml⁻¹ h⁻¹ for initial concentrations of 5, 50 and 500 μg MTBE ml⁻¹, respectively.

Pivateau et al. (Pivateau P., Fayolle F., Vandecasteele J-P. and Monot F. (2001) Appl. Microbiol. Biotechnol. 55: 369-373) have isolated an aerobic bacterial strain CIP I-2052 which is able to use TBA and ETBE as its sole source of carbon and energy. The maximum TBA degradation rate was 35.8±8.5 mg TBA g⁻¹ cell dry mass per hour.

MTBE degradation via TBF was reported in “In Situ and On-Site Bioremediation, The Sixth International Symposium” (Jun. 4-7, 2001, San Diego, Calif.). Martinez-Prado et al. described in a Platform abstract a strain of Mycobacterium vaccae which is capable of degrading MTBE co-metabolically via TBA but could not use MTBE as its sole source of carbon and energy. Hyman et al. described strain VB-1 that has tentatively been identified as a Variovorax strain and is capable of degrading MTBE co-metabolically after growth on aromatic compounds found in gasoline. Soon-Woong et al. presented a poster describing butane-grown microorganisms which were also capable of co-metabolically degrading MTBE via TBF. However, no bacterial strain capable of using MTBE as its sole source of carbon and energy was described.

Thus, as contaminations by fuel and its additives, such as fuel oxy-genating ethers, in soils and groundwater cannot be completely avoided and biodegradation methods are still rather ineffective and slow, there is still a need for more effective and faster biological processes in which these contaminants are degraded. New organisms having an increased capacity to degrade ethers would also be needed. The present invention now provides means for filling these needs.

### SUMMARY OF THE INVENTION

The present invention resides in finding Variovorax strains, which are capable of degrading ethers and their degradation products and even capable of using MTBE as their sole source of carbon and energy. These strains enable fast and efficient degradation of ethers and in their degradation products.

The present invention provides a Variovorax strain, which is characterized in that it is capable of using methyl tert-butyl ether (MTBE) as its sole source of carbon and energy. Such a strain provides an effective way of biologically degrading fuel oxygenating ethers and their degradation products e.g. in soil and groundwater. The present invention also provides a mixed bacterial population, which is characterized in that it comprises one or more strains of the invention.

The present invention further provides a process for bacterial degradation of ethers and their degradation products, which is characterized by fermenting a solution comprising one or more ethers or their degradation products with a bacterial population comprising one or more Variovorax strains capable of using methyl tert-butyl ether (MTBE) as their sole source of carbon and energy.

Further the present invention relates to the use of one or more Variovorax strains of this invention in purifying contaminated soils and water.

### BRIEF DESCRIPTION OF THE DRAWINGS

FIG. 1 illustrates the MTBE degradation pathway of the bacterial strains of this invention. X designates a carrier for protons [H].

### DETAILED DESCRIPTION OF THE INVENTION

The Variovorax strains of the present invention are capable of degrading ethers and their degradation products and using MTBE as their sole source of carbon and energy. The ethers to be degraded can be any ethers, either linear or branched. The ethers are preferably fuel oxygenating ethers, such as ethyl tert-butyl ether (ETBE), tert-amyl methyl ether (TAME), diisopropyl ether (DIPE), diethylether (DEE) and MTBE. The degradation products of ethers include all the compounds that may be found as intermediates in the degradation pathway beginning from the ether and ending finally via the central metabolism in carbon dioxide. The degradation products preferably are degradation products of fuel oxygenating ethers, such as tertiary alcohols. Some degradation intermediates, such as TBA, which is a tertiary alcohol, can also be used as the sole source of carbon and energy by the strain.

It has surprisingly been found that the bacteria of the invention are able to degrade MTBE and TBA much more effectively than any microorganism of the prior art. The Variovorax strains of this invention preferably belong to the species Variovorax paradoxus. Two strains of Variovorax paradoxus, Variovorax paradoxus JV-1 and Variovorax paradoxus CL-3, have been deposited with Deutsche Sammlung von Mikroorganismen und Zellkulturen GmbH (DSMZ, Braunschweig, Germany) with deposit numbers DSM 14357 and DSM 14433, respectively. Strain JV-1 is able to metabolize at least 20 milligrams, preferably at least 60 milligrams and most preferably at least 80 milligrams of MTBE per gram of dry cells per hour. Strain CL-3 is able to metabolize at least 80 milligrams, preferably at least 100 milligrams of TBA per gram of dry cells per hour. Even though this application concentrates on the ability of the bacteria to degrade fuel oxygenating ethers and their degradation products, other bioremediative processes, such as degradation of aromatics or petroleum hydrocarbons, are not excluded.

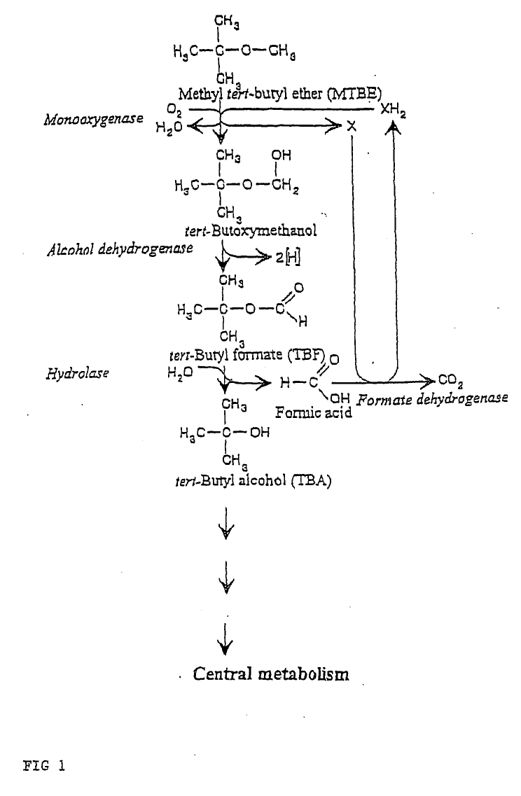

The metabolic pathway of MTBE and TBA degradation was studied by gas chromatography mass spectrometry (GC-MS) and feeding experiments. FIG. 1 illustrates the proposed pathway. TBF and TBA were detected by (GC-MS) as transient intermediates, which accumulated in the culture fluid during growth of the strains on MTBE. The kinetics of metabolite formation demonstrates that TBF accumulation precedes TBA accumulation. Tert-butoxymethanol, which could not be detected in this study, is predicted to be an instable intermediate between MTBE and TBF. TBA and formate together induced MTBE degradation. Thus, it is proposed that formic acid should be cleaved from TBF, and degraded into CO₂. The released hydrogen then reduces a carrier (X), which enhances MTBE breakdown.

In one embodiment of the invention strain JV-1 is used in a process for degrading MTBE and its degradation products in a solution. In another embodiment of the invention strain CL-3 is used for degrading TBA and its degradation products in a solution.

In another embodiment of the invention strains JV-1 and CL-3 are used together in order to degrade fuel oxygenating ethers and their degradation products. A co-culture of the strains is advantageous in degradation processes as strain JV-1 is a very effective MTBE degrader at the beginning of the pathway and strain CL-3 is very effective in degrading TBA, which is a degradation intermediate of MTBE. Effective degradation of TBA is important, because accumulation of TBA could otherwise inhibit the very first steps of the pathway.

The process of the invention is especially suitable for degradation of MTBE and/or TBA containing solutions.

The strains of the invention are suitable for use in bioremediation of solutions in a large-scale reactor. Solutions to be bioremediated can be any aqueous solutions such as sludge of municipal waste-water, industrial waste water or contaminated ground water or any other contaminated water. Preferably the reactor is an aerobic bioreactor with a fixed carrier, to which the microorganisms can attach. Preferably a mixed culture comprising one or more bacterial strains of the invention is used. A mixed culture of various strains is advantageous as there are several different contaminants in water and sludges. Thus many different degradation processes are needed in order to reach an acceptable degradation level of all contaminants. The other bacteria or other microorganisms contained in the mixed population are preferably derived and enriched from water purification processes, e.g. from active sludge.

In still another embodiment of the invention ethers and their degradation products are extracted with an aqueous solution from contaminated soils, such as soils near gas stations and then bioremediated according to the invention.

The solution to be processed in order to degrade contaminating agents can be any aqeuous solution, such as contaminated groundwater, sludge or water collected from contaminated soils. Contaminated soil can be purified, provided that there is enough moistness to allow the microorganisms to live and function. Preferably moistness of the soil is collected to a reactor to ensure optimal conditions for microorganisms to degrade the contaminants. Alternatively the moistness is circulated from the soil to the reactor and back to the soil, several times if needed, in order to ensure that the contaminants in the soil are reduced toan environmentally acceptable level. This embodiment is especially useful when the soil is contaminated with e.g. ethers, which have high water solubility.

The following examples are provided for illustration purposes only and they are not intended to limit the scope of the present invention.

### EXAMPLES

### Example 1

### Isolation and Characterization of the Strains

Strains of the invention were isolated from an active sludge by selective enrichment with MTBE as the sole source of carbon and energy. MTBE (10 μl) and 50 ml of sludge were added to a 1-litre gas-tight flask with 50 ml of CLM medium (1 g K₂HPO₄.3H₂O, 0.25 g NaH₂PO₄.2H₂O, 0.1 g (NH₄)₂SO₄, 0.05 g MgSO₄.7H₂O and Ca(NO₃)₂.4H₂O in 1 litre of distilled or deionized water) containing 10 mg l⁻¹ of yeast extract and incubated stationary at 22° C. After disappearance of the substrate (four months) more MTBE was added. After six months 40 ml of this mixed enrichment culture was suspended in 60 ml of CLM medium containing yeast extract, trace elements and MTBE (10 μl) and incubated stationary at 22° C. (one month), diluted 1:5 (vol/vol) with CLM medium containing MTBE (10 μl/100 ml), trace elements and vitamins and incubated until visibly turbid. The culture, designated as CL-EMC-1, was maintained with transfer intervals of 1.5-2 months using MTBE as the sole source of carbon and energy. An attempt to isolate strains using MTBE or TBA as their sole source of carbon and energy failed at this state. After 3 years the mixed enrichment culture CL-EMC-1 was adapted to 200 mg l⁻¹ of MTBE by periodic (15 days) reinoculations for 4 months.

This culture, which utilized MTBE as the sole source of carbon and energy up to 1.5 g l⁻¹, was now plated onto CLM agar with MTBE. Isolated colonies were tested for the ability to grow in CLM agar with MTBE. Colonies grown on the plates were streaked pure by serial dilutions of single colonies on CLR agar (1 g Soy pepton, 0.2 g trypton and 0.2 g yeast extract in 1 litre of CLM medium with 1.5 to 2.0% (wt/vol) of Bacto-Agar, Difco Laboratories, Detroit, USA). One isolated pure strain was designated JV-1 and it utilized MTBE as its sole carbon and energy source.

20 ml of CL-EMC-1 culture was added to a 1-litre gas-tight flask with 80 ml of CLM medium containing 0.05 g l⁻¹ of TBA, trace elements and vitamins and incubated stationary at 22° C. After disappearance of the substrate (two weeks) more TBA was added. After a month 20 ml of this culture was diluted 1:10 (vol/vol) with CLM medium containing TBA (0.1 g l⁻¹) and incubated on a gyratory shaker at 152 rpm at 28° C. until turbid (7 days). This procedure was repeated three times. Then the culture was plated on CLM agar containing 0.05 g l⁻¹ of TBA at 22° C. Isolated colonies were tested for the ability to grow in CLM medium with TBA as the sole source of carbon and energy. Colonies grown on the plates were streaked pure by serial dilutions of single colonies on CLR agar. One isolated pure strain, designated CL-3, utilized TBA up to 7 μg l⁻¹.

Two isolated strains, JV-1 and CL-3 were identified by DSMZ (Deutsche Sammlung von Mikroorganismen und Zellkulturen GmbH, Braunschweig, Germany). Both the strains have the characteristics of the species Variovorax paradoxus. They are gram negative bacteria having rod like cells, width 0.5-0.7 μm and length 1.5-3.0 μm. The partial sequences of the 16SrDNA show a similarity of 99.3% to Variovorax, the other similarities being much lower. The profiles of the cellular fatty acids and the data of the physiological tests clearly confirm this result. The results of the physiological tests are presented in Table 1 below:

TABLE 1

| Reaction | Strain JV-1 | Strain CL-3 |
|---|---|---|
| Gram reaction | − | − |
| Lysis by 3% | + | + |
| Aminopeptidase (Cerny) | + | + |
| Oxidase | weak | + |
| Catalase | + | + |
| Pigmentation | yellow | yellow |
| Motility | +weak | no data |
| Flagella | + | no data |
| Growth at 30° C. | + | + |
| ADH | − | − |
| Urease | + | + |
| Hyrdolysis of gelatine | − | − |
| Autotrophic growth | − | − |
| Nitrareduction | − | − |
| Denitrification | − | − |
| Utilization of | | |
| glucose | + | + |
| phenylacetat | + | + |
| citrat | + | + |
| malat | + | + |
| arabinose | + | + |
| gluconat | + | + |
| adipat | weak | weak |
| trehalose | + | no data |
| malonate | + | + |
| L-mandelate | − | − |
| trans-aconitate | + | + |
| p-hydroxy-benzoate | + | + |
| L-histidine | + | + |
| etanolamine | − | − |
| mannitol | + | + |
| fructose | + | + (D-fructose) |
| salicine | + | + |
| sachharose | − | − |
| arabitol | + | + |
| inositol | + | + |
| 2-ketoglukonate | + | + |
| m-tartrate | + | + |
| citraconate | no data | + |
| acetat | no data | + |
| sebacinat | no data | + |
| pimelat | no data | + |
| D-xylose | no data | + |
| Hydrolysis of esculine | − | − |

Growth of the strains JV-1 and CL-3 on butane was studied in 2 liter bottles containing 0.2 liter of the minimal salts medium with trace elements and vitamins as described in the following Example 2. The bottles were sealed with screw caps fitted with Teflon-lined silicone septa. 50 ml, 100 ml and 200 ml volumes of butane (99% of purity) were added to the bottles as an overpressure. The strains JV-1 and CL-3 were incubated parallelly stationarily and on a gyratory shaker (152 rpm) at 22° C. after inoculation at an initial absorbance at 540 nm of 0.1 (cell dry weight of 30 mg/l). No growth was detected in the media with butane during five months of incubation in any bottles (absorbance at 540 nm less than 0.1).

### Example 2

### MTBE and TBA Degradation in Laboratory Scale

Growth of the strains JV-1 on MTBE and CL-3 on TBA was studied in laboratory conditions in 2-liter gas-tight flasks with Teflon-wrapped stoppers, containing 0.2 litre of medium. The composition of the minimal salts medium used for the enrichment and cultivation of bacteria of the invention was as follows (grams per litres of distilled or deionised water): K₂HPO₄.3H₂O, 1; NaH₂PO₄.2H₂O, 0.25; (NH₄)₂SO₄, 0.1; MgSO₄.7H₂O, 0.05; Ca(NO₃)₂.4H₂O, 0.02; FeCl₃.6H₂O, 0.002, pH 7.0-7.3. The medium also contained the following elements (milligrams per litre): H₃BO₃, 2; FeSO₄.7H₂O, 2; Na₂SeO₃.5H₂₀, 1; Na₂MoO₄.2H₂₀, 1; CoCl₂.6H₂O, 1; MnSO₄.2H₂O, 0.5; ZnSO₄.7H₂O, 0.5; AlCl₃.6H₂O, 0.05; NiCl₂.6H₂O, 0.02; CuSO₄.7H₂O, 0.01, pH 7.0-7.3. The medium was sterilized 20 min at 121° C. Stock solution of vitamins was as follows (milligrams per liter of distilled or deionised water): Riboflavin, 10; Nicotinic acid, 5; Ca-panthotenate, 5; Thiamine, 5; Folic acid, 2; Pyridoxine hydrochloride, 1; Cyanocobalamin, 1; Biotin, 1. The solution of vitamins (10 ml) was filter-sterilized and added to an autoclaved cooled medium. The inoculation density for strain JV-1 was 1.1×10⁶ to 1.2×10⁷ and for strain CL-3 1.6×10⁶ to 1.1×10⁷ cells per millilitre of medium. An appropriate source of carbon (MTBE or TBA) was added. The strain JV-1 was incubated stationarily at 22° C. and the strain CL-3 was incubated on a gyratory shaker at 28° C.

### Example 3

### Gas Chromatography Mass Spectroscopy Studies on the Novel Pathway

Culture samples obtained from Example 2 were analyzed for MTBE, TBF and TBA employing a gas chromatography mass spectrometry (GC-MS) with HP 6890 gas chromatograph equipped with HP 5973 mass selective detector and PONA crosslinked methylsiloxane capillary columns (50 m by 0.2 mm; 0.5 μm film thickness, Agilent Technologies, U.S.A.). The oven temperature was held at 35° C. for 15 min, followed by an increase at 10° C. min¹ to 70° C., held at this temperature for 3 min and then increase at 20° C. min¹ to 250° C. and held at this temperature for 5 min. The carrier gas (helium) was maintained at a constant column flow of 0.5 ml min¹. Samples were analyzed by electron ionization (70 eV) with full scan monitoring (m/z=25 to 200). Interpretation of mass spectra for identification of compounds was performed by autenthic standards and database in GC-MS software (Wiley 275).

### Example 4

### MTBE Degradation in Laboratory Scale and Comparison to Prior Art

Strain JV-1 was grown under the above-mentioned conditions using MTBE as the sole source of carbon and energy. It was shown that the strain was able to degrade MTBE remarkably faster than strain PM1 reported by Hanson et al, which is the best MTBE degrading bacteria known from prior art. The results of the experiment and comparison between JV-1 and PM1 are shown below in Table 2.

TABLE 2
Comparison of strain Variovorax paradoxus JV-1
with strain PM1 capable of MTBE degradation

| Parameters | Variovorax paradoxus JV-1 | PM1* |
|---|---|---|
| Growth rate on MTBE, h⁻¹ | 0.03 ± 0.004 | No data |
| Growth yield, g (cells dry | 0.47 ± 0.03 | 0.18 ± 0.06 |
| weight) g of MTBE used | | |
| Degradation rate, mg MTBE | 88 ± 14 | 0.07-3.56^{#} |
| g of dry cells h⁻¹ | | |
| MTBE degrading | Up to 1.5 | 0.005-0.5 |
| concentration, gl⁻¹ | | |
| Initial biomass concentration | 3-15 | — |
| for MTBE utilization, mg of | | |
| dry cells liter⁻¹ | | |
| or | | |
| Inoculation density, number | 1.1 × 10⁶-1.2 × 10⁷ | 2 × 10⁶ |
| of cells ml⁻¹ | | |

*= measured by Hanson et al.
^{#}= initial rates of MTBE degradation by 2 × 10⁶ cells ml⁻¹ were 0.07, 1.17 and 3.56 μg ml⁻¹ h⁻¹ for initial concentrations of 5, 50 and 500 μg MTBE ml⁻¹ respectively.

### Example 5

### TBA Degradation in Laboratory Scale and Comparison to Prior Art

Strain CL-3 was grown under the above-mentioned conditions using TBA as the sole source of carbon and energy. It was shown that the strain was able to degrade TBA remarkably faster than strain CIP 1-2052 (reported by Pivateau et al.), which is the best TBA degrading bacteria known from prior art. The results of the experiment and comparison between CL-3 and CIP 1-2052 are shown below in Table 3.

TABLE 3
Comparison of strain Variovorax paradoxus CL-3 with strain
Burkholderia cepacia CIP I-2052 capable of TBA degradation.

| | Variovorax | Burkholderia |
|---|---|---|
| | paradoxus | cepacia |
| Parameters | CL-3 | CIP I-2052* |
| Maximum growth rate | 0.042 ± 0.005 | 0.032 ± 0.004 |
| on TBA, h⁻¹ | | |
| Growth yield, g (cells | 0.59 ± 0.04 | 0.54 ± 0.02 |
| dry weight) g of TBA | | |
| used | | |
| Maximum degradation | 99.5 ± 10.5 | 35.8 ± 8.5 |
| rate, mg TBA g of | | |
| dry cells h⁻¹ | | |
| TBA degrading | Up to 7 | Up to 6 |
| concentration, | | |
| gl⁻¹ | | |
| Initial biomass | 3-15 | 47 |
| concentration | | |
| for TBA utilization, | | |
| mg of dry | | |
| cells liter⁻¹ | | |
| or | | |
| Inoculation density, | 1.6 × 10⁶-1.1 × 10⁷ | No data |
| number of cells ml⁻¹ | | |
| MTBE degradation | 0.41 | Could not |
| rate, mg MTBE g⁻¹ of | | degrade |
| dry cells h⁻¹ | | MTBE |

*= measured by Pivateau et al.

### Example 6

### Large Scale Bioremediation

The strains of the invention were tested in a large-scale experiment during three years. MTBE contaminated ground water was incubated in an aerobic bioreactor of 100 m³ provided with a fixed carrier. The reactor was inoculated with mixed bacterial culture comprising strains JV-1 and CL-3 and other bacteria isolated from activated sludges. The flow rate was 35 m³ of groundwater per day. The average temperature of the water was 16° C., but the inventors have demonstrated that MTBE can be degraded by strain JV-1 even at 8° C. The reactor was operating 3 years. Remarkable reduction in MTBE and some other organic contaminating agents was observed. The results of the experiment are as follows:

TABLE

Concentration of
contaminants in
water (μl)

| Organic | Before | After |
|---|---|---|
| contaminating agent | treatment | treatment |
| MTBE | 5-11 | <1 |
| Benzene | 1-3 | <1 |
| Toluene | 1-12 | <1 |
| Ethylbenzene | 1-4 | <1 |
| m/p-Xylene | 2-4 | <1 |
| o-Xylene | 2-5 | <1 |
| 1,3,5-Trimetylbenzene | 2-10 | <1 |
| 1,2,4-Trimetylbenzene | 2-12 | <1 |
| Naphthalene | 3-35 | <0.05 |
| Acenaphthene | 0.1-7 | <0.05 |
| Fluorene | 0.1-5 | <0.05 |
| Phenanthrene | 0.1-6 | <0.05 |
| Anthracen | 0.1-0.2 | <0.05 |

1. A Variovorax strain, said Variovorax strain being capable of using methyl tert-butyl ether (MTBE) as its sole source of carbon and energy.

2. The strain of claim 1, wherein the strain belongs to the species Variovorax paradoxus.

3. The strain of claim 2, wherein the strain is Variovorax paradoxus JV-1, which has deposit number DSM 14357.

4. The strain of claim 2, wherein the strain is Variovorax paradoxus CL-3, which has deposit number DSM 14433.

5. A mixed bacterial population, comprising one or more of the strains of claim 1.

6. A process for bacterial degradation of ethers and their degradation products, comprising the steps of fermenting a solution comprising one or more ethers or their degradation products with a bacterial population comprising one or more Variovorax strains capable of using methyl tert-butyl ether (MTBE) as their sole source of carbon and energy.

7. The process of claim 6, wherein the fermenting is performed with a bacterial population comprising one or more strains belonging to the species Variovorax paradoxus.

8. The process of claim 7, wherein the fermenting is performed with a bacterial population comprising Variovorax paradoxus JV-1, which has deposit number DSM 14 357.

9. A process of claim 7, wherein the fermenting is performed with a bacterial population comprising Variovorax paradoxus is CL-3, which has deposit number DSM 14433.

10. The process of claim 6 wherein the fermenting is of fuel oxygenating ethers or their degradation products

11. The process of claim 10, wherein the fermenting is of one or more of the following compounds: ethyl tert-butyl ether (ETBE), tert-amyl methyl ether (TAME), diisopropyl ether (DIPE), diethylether (DEE), methyl tert-butyl ether (MTBE) and tert-butyl alcohol (TBA) containing solutions.

12. A process of claim 11, wherein the fermenting is of methyl tert-butyl ether (MTBE) and tert-butyl alcohol (TBA) containing solutions.

13. A method for purifying contaminated soil or water, comprising using a Variovorax strain, said Variovorax strain being capable of using methyl tert-butyl ether (MTBE) as its sole source of carbon and energy.

14. A mixed bacterial population, comprising one or more of the strains of claim 2.

15. A mixed bacterial population, comprising one or more of the strains of claim 3.

16. A mixed bacterial population, comprising one or more of the strains of claim 4.

17. The process of claim 7, wherein the fermenting is of fuel oxygenating ethers or their degradation products.

18. The process of claim 8, wherein the fermenting is of fuel oxygenating ethers or their degradation products.

19. The process of claim 9, wherein the fermenting is of fuel oxygenating ethers or their degradation products.
